(19) United States
(12) Patent Application Publication
(10) Pub. No.: US 2026/0154792 A1
(43) Pub. Date: Jun. 4, 2026

(54) UNSUPERVISED MACHINE LEARNING PARADIGM TO CONTROL CARDIAC AND RESPIRATION RELATED MOTION ARTIFACTS

(71) Applicant: St. Jude Medical, Cardiology Division, Inc., St. Paul, MN (US)

(72) Inventors: Patrick K. Kasi, Woodbury, MN (US); Hana Y. Boudlali, Blaine, MN (US); Joseph Mathew, St. Paul, MN (US); Rachel S. Keating, Shoreview, MN (US); Daniel S. Stein, Lino Lakes, MN (US); Scott M. Seieroe, Eden Prairie, MN (US); Anthony Hill, Rochester, MN (US); Kristi M. Tanouye, Minneapolis, MN (US); Ryan M. Albu, Minneapolis, MN (US)

(21) Appl. No.: 19/407,229

(22) Filed: Dec. 3, 2025

Related U.S. Application Data
(60) Provisional application No. 63/727,884, filed on Dec. 4, 2024.

(51) Int. Cl. G06T 5/70; A61B 1/04; A61B 5/06; G06T 7/00

(52) U.S. Cl. CPC G06T 5/70; A61B 1/04; A61B 5/068; G06T 7/0012; G06T 2207/30048

(57) ABSTRACT

A method of filtering unwanted motion related artifacts from received sensor-based location data includes receiving unfiltered location data and calculating a plurality of spatio-temporal components associated with the unfiltered location data. The method further includes comparing each spatio-temporal component to the unfiltered location data and updating a weight value based on the comparison of each spatio-temporal component to the unfiltered location data to generate a dynamically updated set of weights. Motion corrected location data is calculated based on the dynamically updated set of weights and the plurality of spatio-temporal components.

### CROSS-REFERENCE TO RELATED APPLICATIONS

This application claims the benefit of and priority to U.S. provisional application 63/727,884, titled “UNSUPERVISED MACHINE LEARNING PARADIGM TO CONTROL CARDIAC AND RESPIRATION RELATED MOTION ARTIFACTS”, filed Dec. 4, 2024, the contents of which are incorporated by reference herein.

### TECHNICAL FIELD

The subject matter disclosed herein relates to localization of sensors in medical devices and, in particular, to filtering motion artifacts related to cardiac motion and respiration.

### BACKGROUND

Numerous technologies have been developed to permit the determination of the location of a catheter inside of a beating heart. For example, impedance-based localization uses transthoracic electric field applied via standard—left to right, chest to back, and neck to leg—body surface electrodes (patches). Three electrical currents are applied to the patches, resulting in a voltage gradient across the heart. Electrodes located on the catheter measure voltages arising from the three injected currents, which are then processed to resolve the X, Y and Z axis coordinates defining the position of the electrodes and more generally the catheter.

A common use of the catheter position is for displaying a representation of the catheter with respect to generated cardiac anatomical models that guide diagnosis and therapy administration or other imaging of a region of interest in which the catheter is located. However, patient respiration and cardiac activity can make the displayed catheter appear to “move” with respect to the acquired cardiac geometries (or imaging), which are static. In order to reduce the apparent motion of the catheter with respect to these static geometries (or imaging) and provide a clinician with a more stable view, it is known to use motion compensation to correct for the effects introduced by patient respiration and cardiac activity.

A typical compensation approach may include using external patches/sensors located on the patient's chest to estimate the respiration contribution to catheter position of a patient, and subtracting that contribution from catheter electrodes within the heart to reduce effects of motion artifacts caused by respiration. However, this assumes that the contribution of respiration motion is uniform across the heart and that the captured external signal represents the right amount of distortion captured by indwelling catheter electrodes. In addition, if the respiration patterns of the patient changes during a procedure (due to anesthesia administration or a patient's breathing patterns change) the already estimated magnitude of cardiac motion does not necessarily reflect the current state of cardiac motion artifact—and this compromises the effectiveness of the established respiration compensation scheme leading to poor outcomes. An alternative approach registers anatomical model and electroanatomic map data are only at end of diastole using respiratory gating at end expiration. This invalidates data points outside the gating window—that may be valuable to a physician or if deliberated added to will have respiration artifact. Other approaches incorporate imaging modalities such as ultrasound. Other alternatives use multiband filters under the assumption that respiration variability—inter-patient and intra-patient—is relatively unchanged around a respiratory frequency of 0.3 Hz. These multiband filtering techniques do not account for changes in respiration signals. Moreover, they cannot account for cardiac motion at all and still rely on external 12-lead ECG recording to compensate for cardiac motion—which is more variable across patients. These techniques assume that the physiological signals (cardiac motion components, respiration components) are stationary and linear. Changes in a patient's breathing pattern or heart rate result in errors in the filtering.

Therefore, it would be beneficial to develop a system and method that is capable of accurately filtering motion artifacts caused by respiration and/or cardiac activity, under all clinical scenarios.

### SUMMARY

According to one aspect, a method of filtering unwanted motion related artifacts from unfiltered sensor-based location data collected from a cardiac-based medical device includes decomposing the unfiltered sensor-based location data into a plurality of spatio-temporal components and calculating for each spatio-temporal component a correlation output based on calculated correlation between the spatio-temporal component and the unfiltered sensor-based location data. Weight values are updated based on the correlation output calculated for each spatio-temporal component to generate a dynamically updated set of weights. Motion corrected location data (i.e., the desired signal) is calculated based on the dynamically updated set of weights and the plurality of spatio-temporal components. The unfiltered sensor-based location data may be obtained from sensors on a medical device. The motion corrected location data may be used to determine the location of the medical device.

The unfiltered location data may be impedance-based location data received from one or more electrodes located on the medical device.

The unfiltered location data may be magnetic based location data received from one or more magnetic sensors located on the medical device.

The dynamically updated set of weights may be iteratively updated based on new spatio-temporal components calculated in response to updated unfiltered location data.

The plurality of spatio-temporal components may be calculated with respect to unfiltered location data collected with respect to a window of length N, wherein a time lag between the unfiltered location data and the motion corrected location output is less than a threshold value.

The spatio-temporal components may be intrinsic mode functions calculated utilizing empirical mode decomposition (EMD) techniques.

Updating a weight value based on the correlation output calculated for each spatio-temporal component to generate a dynamically updated set of weights may include setting the weight value to a value of ‘1’ if the correlation output calculated between a particular spatio-temporal component and the unfiltered location data is greater than a threshold value K and setting the weight value to a value of ‘0’ if the correlation output calculated between a particular spatio-temporal component and the unfiltered location data is less than a threshold value κ.

The plurality of spatio-temporal components cᵢ(n) may be organized into a N×M matrix C(n), wherein Nis the window length and Mis the number of spatio-temporal components calculated for each epoch.

The plurality of weights may be organized into a M×N matrix W(n), wherein Mis the number of weights calculated for each epoch and N is the window length, wherein the motion corrected location data is calculated by multiplying the spatio-temporal matrix C(n) by the weight matrix W(n) and taking the elements located on the diagonal of the resulting matrix as the motion corrected location data.

According to another aspect, a localization system for an intracardiac medical device includes a localization module that receives sensor inputs from sensors located on a medical device and converts the sensor inputs into unfiltered location data and a motion compensation module that receives the unfiltered location data from the localization module and generates in response motion corrected localization data. The motion compensation module comprises a first stage that decomposes the unfiltered location data into a plurality of spatio-temporal components and a second, unsupervised machine learning stage that dynamically updates a plurality of weights based on calculated correlations between each of the plurality of spatio-temporal components and the unfiltered location data, wherein the motion corrected localization data is calculated by multiplying the plurality of spatio-temporal components by the dynamically updated set of weights.

The unfiltered location data may be based on sensor input provided by one or more electrodes located on the intracardiac medical device.

The unfiltered location data may be based on sensor input provided by one or more magnetic sensors location on the intracardiac medical device.

The dynamically updated set of weights may be iteratively updated based on new spatio-temporal components calculated in response to updated unfiltered location data.

The plurality of spatio-temporal components may be calculated with respect to unfiltered location data collected with respect to a window of time N, wherein a time lag between the unfiltered location data and the motion corrected location output is less than a threshold value.

The spatio-temporal components may be intrinsic mode functions calculated utilizing empirical mode decomposition (EMD) techniques.

Updating a weight value based on the correlation output calculated for each spatio-temporal component to generate a dynamically updated set of weights may include setting the weight value to a value of ‘1’ if the correlation output calculated between a particular spatio-temporal component and the unfiltered location data is greater than a threshold value K and setting the weight value to a value of ‘0’ if the correlation output calculated between a particular spatio-temporal component and the unfiltered location data is less than a threshold value K.

The plurality of spatio-temporal components cᵢ(n) may be organized into a N×M matrix C(n), wherein Nis the window length and Mis the number of spatio-temporal components calculated for each epoch.

The plurality of weights may be organized into a M×N matrix W(n), wherein Mis the number of weights calculated for each epoch and Nis the window length, wherein the motion corrected location data is calculated by multiplying the spatio-temporal matrix C(n) by the weight matrix W(n) and taking the elements located on the diagonal of the resulting matrix as the motion corrected location data.

In addition, the disclosure provides an unsupervised machine learning algorithm to dynamically remove motion related artifacts (e.g., cardiac, respiration, or other sources of noise) from the displayed location of the device. In particular, the disclosed system/method relies only on local sensor data (e.g., local electrode data, local magnetic data) and does not require data from other sensors (e.g., surface patch sensors to detect respiration) to detect and compensate for motion related artifacts.

The system/method described herein utilizes a two-part method to dynamically remove motion related artifacts. The first part receives as an input the unfiltered location data x(n) of a given sensor and decomposes the unfiltered location data x(n) into spatio-temporal components. For example, empirical mode decomposition (EMD) may be utilized to decompose the unfiltered location data into a plurality of intrinsic mode function components cᵢ(n). Each intrinsic mode function cᵢ(n) represents a simple oscillatory mode. At least one of the oscillatory modes calculated will be related to cardiac motion, at least one of the oscillatory modes calculated will be related to respiration, and at least one of the oscillatory modes calculated will be related to movement caused by the physician (the desired signal, labeled ŷ(n)). In this way, no prior assumptions are made about the received data. Rather, the decomposition of the signal into one or more oscillatory modes allows for changes in the motion artifacts (e.g., changes in breathing, changes in heart rate) to be accounted for in the filtering process. The method is adaptive and data-driven and therefore does not rely on predefined basis functions like Fourier or wavelet transforms.

The second part is an unsupervised machine learning motion artifact removal system to identify the oscillatory modes that should be kept (i.e., those representative of physician-initiated movement) and those oscillatory modes related to motion artifacts (cardiac artifacts, respiratory artifacts, etc.). The unsupervised machine learning algorithm acts to optimize a signal reconstructed from the oscillatory modes as compared to the original signal given one or more constraints. In some embodiments, one of the constraints utilized is a correlation threshold between the oscillatory mode and the original signal.

The unsupervised machine learning is implemented by applying a correlation function to compare each of the plurality of oscillatory modes to the unfiltered signal and setting weights according to the outcome. For example, if the correlation between a particular oscillatory mode and the unfiltered signal is greater than some threshold, then a weight is set to ‘1’. If the correlation between a particular oscillatory mode and the unfiltered signal is less than a threshold, then a weight is set to ‘0’. Give a window of length N, and a number of oscillatory modes M, an M by N weight matrix W(n) is dynamically generated and updated in response to updated inputs.

A reconstructed signal (i.e., free of unwanted motion artifacts) is generated by applying the dynamically created weight matrix W(n) to a matrix comprised of the oscillatory modes collected over the window of length N, as given by equation: ŷ (n)=C(n)W(n). The weights are iteratively updated based on the cross-correlation criterion and thus provides unsupervised machine learning to filter motion artifacts. Benefits of the disclosed system/method include the ability to capture variability: nonlinearities and non-stationarities consistent with physiological systems such as respiration and cardiac motion.

According to another aspect, a method of separating motion artifact signals from desired catheter manipulation signal aims to exploit merits of multi-resolution structure, spatial, and adaptation. The method includes calculating a plurality of spatiotemporal components associated with the unfiltered location data. The method further assesses features of the spatiotemporal components against the unfiltered location data and dynamically updates a weight value for each spatiotemporal component. A weighted sum of spatiotemporal components derives the desired signal—free of unwanted motion artifact.

### BRIEF DESCRIPTION OF THE DRAWINGS

FIG. 1 is a block diagram of a catheter system including sensor localization.

FIG. 2 is a schematic diagram illustrating the various components of catheter sensor motion, including respiration motion, cardiac motion and physician-initiated motion, and the resulting noisy signal received as an output from catheter sensors.

FIG. 3 is a block diagram of the two stages of signal analysis utilized to filter unwanted motion artifacts from unfiltered localization data to generate the desired motion corrected localization data ŷ(n).

FIG. 4 is a flowchart of steps performed by the motion compensation module to calculate motion corrected location data from the unfiltered location data

FIG. 5 is a flowchart of steps performed by the first stage of the signal analysis to generate the intrinsic mode functions.

FIG. 6 is a flowchart of steps performed by the second stage of the signal analysis utilizing unsupervised machine learning to generate the desired noise free output ŷ(n).

FIG. 7 is a graph comparing noisy data received from the catheter sensor and the desired noise free output ŷ(n) generated using the two-stage signal analysis method.

FIG. 8 is a flowchart of steps performed by the motion compensation module to calculate motion corrected location data from the unfiltered location data.

FIG. 9 is a flowchart of steps performed by the motion compensation module to calculate motion corrected location data from the unfiltered location data.

FIG. 10 is a block diagram of the two stages of signal analysis utilized to filter unwanted motion artifacts from unfiltered localization data to generate the desired motion corrected localization data ŷ(n).

### DETAILED DESCRIPTION

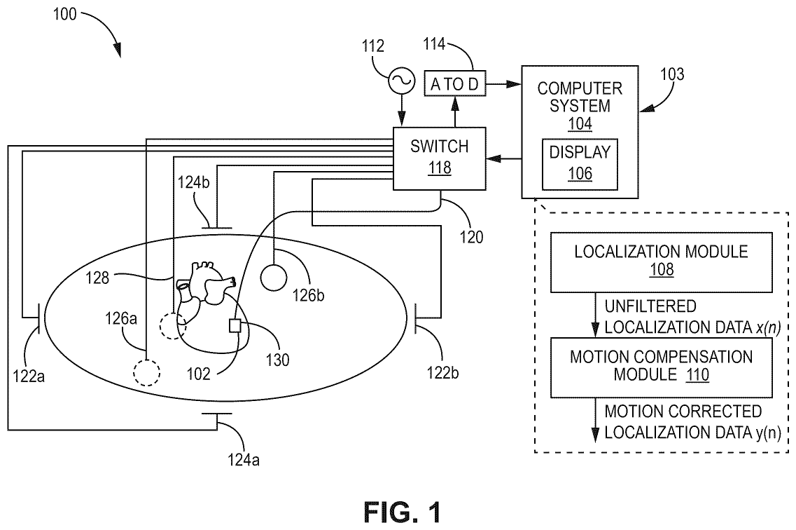

FIG. 1 is a block diagram of a medical device system 100 that includes an intracardiac elongated medical device 120 that includes at a distal end 102 one or more sensors utilized for device localization within the patient's body, including the patient's heart 130. The proximal end of the intracardiac medical device 120 is connected to local system 103, which includes computer system 104 and display 106. Sensed signals provided by the sensors located at the distal end 102 of the medical device 120 are communicated to the computer system 104 for processing to determine the location of the sensor within the patient's body. In some embodiments, the computer system 104 includes memory for storing instructions executed by a processor/controller included as part of the computer system to implement a localization module 108 and a motion compensation module 110.

The localization module 108 implemented by the computer system 104 is configured to receive sensor inputs (e.g., the sensed signals) from the sensors located on the medical device 120 and convert the sensor inputs into unfiltered location data x(n). The unfiltered location data x(n) indicates the position of the sensor with respect to some reference frame. As described in more detail below, the unfiltered location data x(n) includes motion artifacts caused by cardiac motion (i.e., beating of the heart) and respiration of the patient. Display of the unfiltered location data to the physician via display 106—typically overlayed onto an image of the patient's heart-causes the position of the medical device to appear to jump around despite no intentional movement of the device 102 by the physician. That is, the movement attributable to motion artifacts (e.g., cardiac motion, respiratory motion) is not helpful to the physician in understanding the position of the medical device 102 within the patient. To prevent this, the unfiltered location data x(n) is provided to motion compensation module 110 implemented by the computer system 104, which acts to filter the motion artifacts caused by cardiac motion and respiration to generate motion corrected localization data y(n). The motion corrected localization data y(n) is utilized by the computer system 104 to generate a visual output displayed to the physician via display 106. The motion compensation module 110 is described in more detail below. In particular, the motion compensation module 110 does not rely on additional sensors such as surface patch electrodes adhered to the skin of the patient and utilized to monitor respiration to compensate for the effects of cardiac motion and/or respiration. As will be described in more detail below, the motion compensation module 110 comprises: a first stage that is arranged to decompose the unfiltered location data into a plurality of spatio-temporal components; and a second, unsupervised machine learning stage that is configured to dynamically update a plurality of weights based on calculated correlations between each of the plurality of spatio-temporal components and the unfiltered location data, wherein the motion corrected localization data is calculated by multiplying the plurality of spatio-temporal components by the dynamically updated set of weights.

The localization data may be generated by a plurality of different types of localization systems. The embodiment shown in FIG. 1 illustrates an impedance-based localization system that relies on electrodes (not shown) located on the medical device 102 to sense a voltage/impedance field generated across the patient's body by a plurality of surface patch electrodes 122a, 122b, 124a, 124b, 126a, and 126b. Impedance based localization systems include a signal generator 112 that is selectively applied by switch 118 to surface patch electrode pairs (e.g., 122a, 122b) to create a voltage gradient between the pair of electrodes. As the distal end 102 of the intracardiac medical device 120 navigates within the body, electrodes located on the distal end 102 sense a voltage associated with the voltage gradient. The voltage sensed by the electrode is communicated to the computer system 104 via switch 118, low-pass filter 116, and analog-to-digital converter 114, which processes the sensed signal as described above. In other embodiments, other types of localization systems may be utilized to determine the location of the distal end 102 of the medical device 120, either alone or in conjunction with the impedance-based localization systems. For example, magnetic-based localization systems utilizing an external magnetic field generator and one or more magnetic sensors located on the distal end 102 of the medical device 120 may also be utilized to sense signals utilized by the computer system 104 to generate unfiltered localization data. Regardless of the type of sensor utilized to capture localization data, the issues regarding motion artifacts remain the same as do the benefits of motion compensation provided by motion compensation module 110.

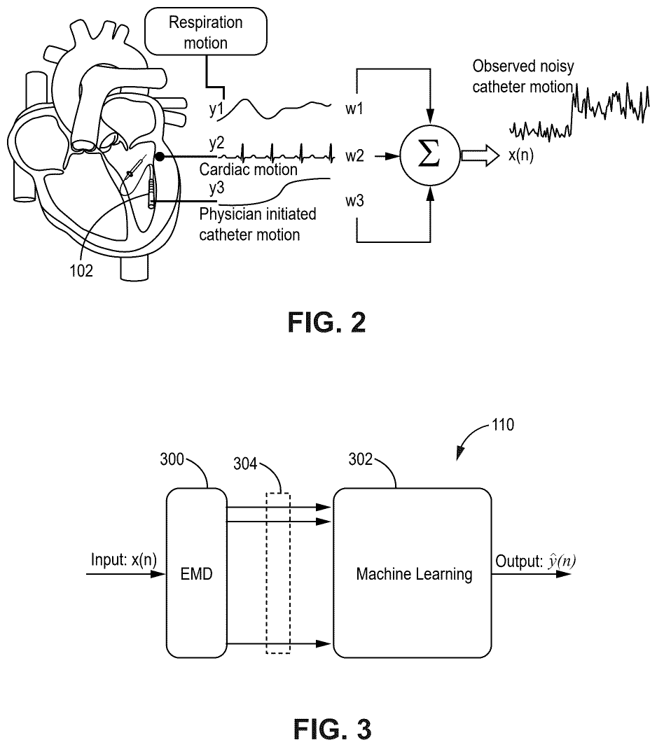

FIG. 2 is a schematic diagram illustrating the various components of motion associated with sensor data received from the distal end 102 of the medical device 120, including respiration motion, cardiac motion and physician-initiated motion, and the resulting noisy signal received as an output from catheter sensors. For the sake of simplicity, the motion associated with the medical device is illustrated as three separate motion signals, respiration motion y1, cardiac motion y2, and physician-initiated motion y3. The sum of the three separate motion signals is the signal sensed by the sensor and is referred to as the unfiltered or noisy location data. Displaying the unfiltered location data to the physician via the display would result in the position of the distal end of the catheter jumping around as a result of the motion artifacts (e.g., respiration motion y1 and cardiac motion y2). It is desirable filter the motion artifacts so that the location data displayed to the physician includes only the physician-initiated catheter motion illustrating to the physician how the distal end 102 of the medical device is being moved through the patient.

FIG. 3 is a block diagram of the two stages of signal analysis utilized by the motion compensation module 110 to filter unwanted motion artifacts from the unfiltered location data x(n) to generate the desired noise free output ŷ(n). The motion compensation module 110 includes a decomposition stage 300 and an unsupervised machine learning stage 302. The input provided to the decomposition stage 300 is the unfiltered location data x(n). The decomposition stage 300 decomposes the unfiltered location data x(n) into a plurality of spatio-temporal components 304. In one embodiment, empirical mode decomposition is utilized to decompose the unfiltered location data x(n) into a plurality of intrinsic mode functions (IMFs). Empirical mode decomposition acts to adaptively and locally decompose a non-stationary time series into a sum of intrinsic mode functions (IMF) which represent zero-mean amplitude and frequency modulated components. A benefit of empirical mode decomposition is that it is a lossless operation. That is, superimposing all extracted IMFs together with the remaining residual results in the reconstruction of the original signal without information loss or distortion. The number of IMFs 304 calculated is variable and dependent on the signal provided as an input.

The unsupervised machine learning stage 302 receives the plurality of IMFs as an input and generates as an output motion corrected location data ŷ(n). The unsupervised machine learning stage 302 dynamically modifies a plurality of weights that are applied to the plurality of IMFs to generate the desired motion corrected location data ŷ(n). The weights are continually updated based on a correlation function applied with respect to each IMF and the unfiltered location data x(n). The correlation function is utilized to optimize the weights applied to the plurality of IMFs, wherein the weights act to remove or retain IMF components for inclusion as part of the motion corrected location data ŷ(n).

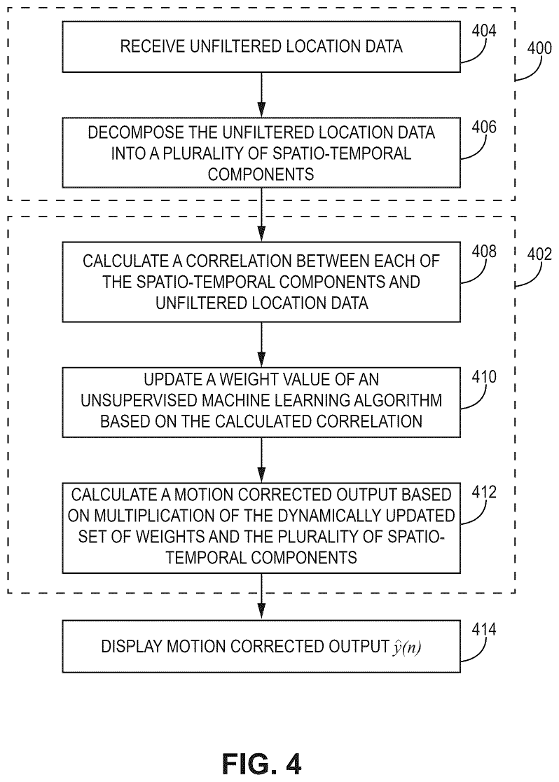

FIG. 4 is a flowchart illustrating steps performed by motion compensation module 110 to remove unwanted motion artifacts from the unfiltered location data. Stage 400 illustrates steps performed by the decomposition stage and stage 402 illustrates steps performed by the unsupervised machine learning phase.

At step 404, the unfiltered location data is received as an input. As described above, the unfiltered location data may be generated by a plurality of different types of localization systems, including for example impedance-based localization systems and magnetic-based localization systems. In some embodiments, the unfiltered location data is pre-processed to remove noise but includes undesirable motion artifacts as well as physician induced motions.

At step 406, the unfiltered location data is decomposed into a plurality of decomposed components. For example, empirical mode decomposition may be utilized to decompose the unfiltered location data into a plurality of spatio-temporal components referred to as intrinsic mode functions (IMFs). In other examples, variational mode decomposition is utilized to decompose the unfiltered location data into a plurality of variational modes, each variational mode representing a distinct spatio-temporal component of the subject's motion.

At step 408, a correlation function result is calculated between each IMF component and the unfiltered location data signal. The output of the correlation function represents a measure of how strongly the respective inputs fluctuate together. The higher the correlation, the more likely the IMF component is representative of physician induced motion. The lower the correlation, the more likely the IMF component is representative of motion induced artifacts.

At step 410, weight values are updated based on the correlation output calculated at step 408. In some embodiments, the weight values are binary values set to ‘0’ to indicate the correlation output between a particular IMF component and the unfiltered location data is low or ‘1’ to indicate the correlation output between the IMF component and the unfiltered location data is high. In this way, a matrix of weights is generated describing the correlation between the unfiltered input and the plurality of IMF components.

At step 412, motion corrected location data ŷ(n) is calculated based on the dynamically updated set of weights and the calculated IMFs. In particular, as described in more detail below the dynamically updated set of weights are stored as a matrix of individual weights corresponding to each of the plurality of IMFs over a corresponding window of time. Likewise, the plurality of IMF components collected over the corresponding window of time are stored as a matrix. Multiplication of the two matrices results in multiplication of each weight with the corresponding IMF component associated with that weight. The result of the multiplication is a matrix representing the motion corrected location data over the given window.

At step 414, the motion corrected location data ŷ(n) is displayed (e.g., via display 106). In some examples, the motion corrected location data ŷ(n) is displayed in the form of an image representing the electrode or in the form of an image representing the medical device including the one or more electrodes.

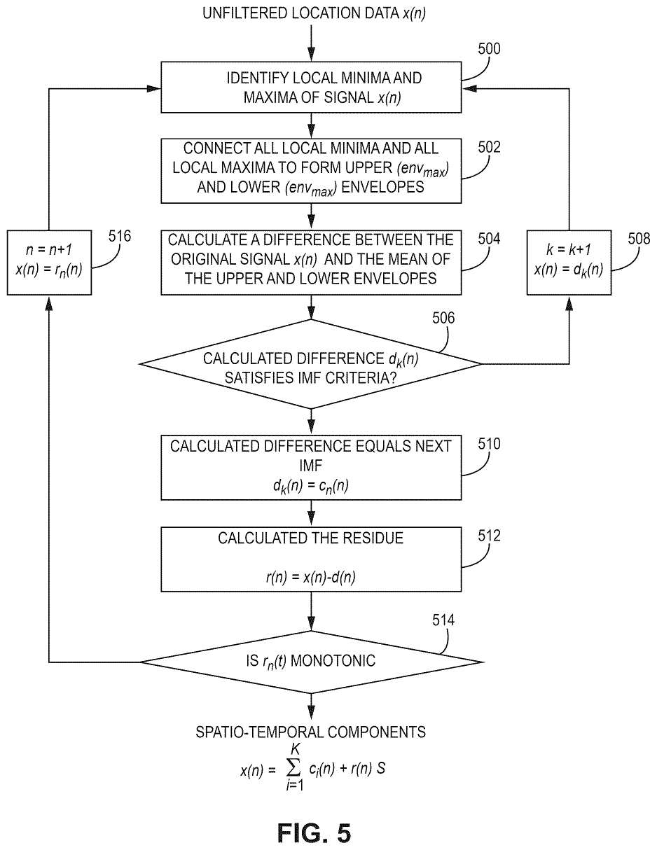

FIG. 5 is a flowchart of steps performed by the first stage (i.e., decomposition stage 300 shown in FIG. 3) of the motion compensation module 110 to decompose the unfiltered location data into a plurality of spatio-temporal components. The unfiltered location data x(n) is received as an input. The value of x(n) is modified as IMFs are extracted from the original signal, but initially x(n) is representative of the unfiltered location data.

At step 500, local minima and maxima are identified within the unfiltered location data x(n). Local minima are defined by the values on either side of the minima being greater than the local minima value. Local maxima are defined by the values on either side of the maxima being less than the local maxima value.

At step 502, all local minima values are connected to form a lower envelope line envₘᵢₙ and all local maxima values are connected to form an upper envelope line envₘₐₓ. For example, in some embodiments cubic spline interpolation may be utilized to construct the upper and lower envelopes.

At step 504, a difference dₖ(n) is calculated between the unfiltered location data x(n) and the mean of the upper and lower envelopes lines envₘₐₓ and envₘᵢₙ. The mean of the upper and lower envelopes is a running mean and is referred to as the low frequency local trend.

At step 506, the calculated difference dₖ(n) is compared to an IMF criteria threshold. If the criteria is not satisfied, then at step 508 k is incremented and the input x(n) is set equal to the calculated difference dₖ(n) for the next iteration. If the criteria is satisfied, then at step 510 the calculated difference dₖ(n) is identified as the next IMF component cx(n).

At step 512, a residue r(n) is calculated by subtracting the calculated difference dₖ(n) from the signal x(n).

At step 514, a determination is made if the calculated residue r(n) is monotonic, indicating no further IMF components may be extracted. If the calculated residue is not monotonic, then at step 516 the time step n is incremented, and the signal x(n) is set equal to the calculated residue r(n) and the process continues at step 500 to identify the next IMF component. If the calculated residue is monotonic, then the process ends as all IMF components cᵢ(n) have been extracted from the unfiltered location data. This is represented by the equation:

$$x ( n ) = {\sum }_{i = 1}^{k} {c}_{i} ( n ) + r ( n ) \qquad ( 1 )$$

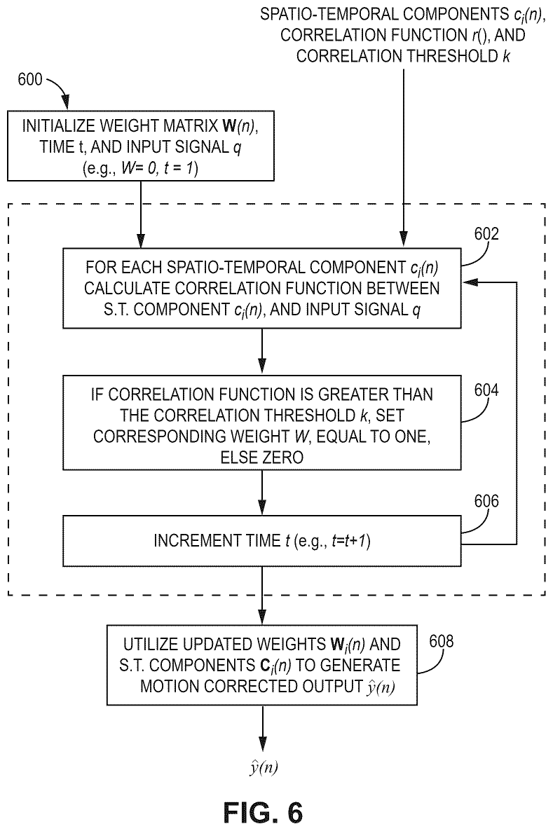

FIG. 6 is a flowchart of steps performed by the second stage (i.e., unsupervised machine learning stage 302 shown in FIG. 3) of the motion compensation module 110 to utilize unsupervised machine learning to generate the desired noise free output ŷ(n) (i.e., motion corrected location data). In general, the second stage acts to dynamically update a set of weights to optimize the correlation between the unfiltered localization data and each of the plurality of spatio-temporal components subject to one or more constraints. The input to the second stage of the signal analysis are the decomposition components Cᵢ(n) calculated during the first stage (as described with respect to FIG. 5), a correlation function r( ) and a correlation threshold k. In some embodiments, the correlation threshold is selected empirically. In some embodiments, the correlation threshold x may be dynamically modified during operation to ensure a non-zero result is provided as an output.

At step 600, the weight matrix W(n), time t, and input signal q are initialized. For example, the weight matrix W(n) may be initialized to all zero values and the time/may be initialized to a time of zero. Initializing the weight matrix W(n) to zero values indicates that none of the decomposition components will be given any weight in identifying the noise free signal ŷ(n). The input signal q may be set equal to the unfiltered localization data x(n).

At step 602, the correlation function r( ) is applied comparing each decomposition component cᵢ(n) to the input signal q. For example, in some embodiments the correlation function r(n) assesses the similarity of the decomposition component cᵢ(n) with the input signal q. In other embodiments, other types of thresholding or statistical operations may be utilized to assess the similarity of the decomposition components cᵢ(n) with the input signal q.

At step 604, the correlation output calculated with respect to each correlation component cᵢ(n) is compared to the correlation threshold K. If the correlation output calculated with respect to a particular correlation component cᵢ(n) is greater than the correlation threshold x then the corresponding weight is set to a value indicating correlation between the particular decomposition component cᵢ(n) and the input signal q (e.g., ‘1’). If the correlation output calculated with respect to a particular correlation component cᵢ(n) is less than the correlation threshold k then the corresponding weight is set to a zero value. In this way, the weight matrix W(n)—in which each matrix position corresponds to a particular decomposition component at a given time t—is populated based on the calculated correlation function. The weight matrix W(n) is an M×N matrix wherein M represents the number of decomposition components and N is the length of the window analyzed for adaptive analysis. The correlation function r( ) represents one of the constraints on the optimization problem, and modification of the correlation threshold κ may be utilized to modify the constraints on the optimization problem. This constraint may be expressed mathematically as follows:

$$\frac{1}{N} {\sum }_{i = 1}^{N} y ( n ) {c}_{i} ( n ) \geq r \qquad ( 2 )$$

In some embodiments, the optimization problem is further subject to a constraint on the lag between the reconstructed motion compensated signal and the original signal, expressed mathematically as follows:

$$τ ( y ( n ) , tr W ( n ) C ( n ) ) \leq t \qquad ( 3 )$$

where τ is the time lag between reconstructed and original signal and t is the threshold time value.

At step 606, time t is incremented (e.g., t=t+1) and the process repeats at step 602 based on updated decomposition components cᵢ(n) and input signals q.

In addition, at step 608 for each updated weight matrix W(n), the updated weight matrix W(n) is applied to the plurality of spatio-temporal components C(n) to reconstruct the motion corrected location data § (n). The plurality of spatio-temporal components C(n) may be organized into a N×M matrix, wherein N is the window length and M is the number of spatio-temporal components calculated for each epoch. The plurality of weights are organized into a M×N matrix W(n), wherein M is the number of weights calculated for each epoch and Nis the window length. In some embodiments, the weight matrix W(n) is multiplied with the decomposition matrix C(n) to generate the desired motion corrected location data given by the following equation:

$$\hat{y} ( n ) = tr [ C ( n ) W ( n ) ] \qquad ( 4 )$$

wherein the operator “tr” indicates the trace of the resulting square matrix and may include the sum of the elements located on the diagonal of the resulting square matrix.

The motion corrected localization data ŷ(n) may be utilized to generate the display of the medical device on the display 106 (shown in FIG. 1) for the physician.

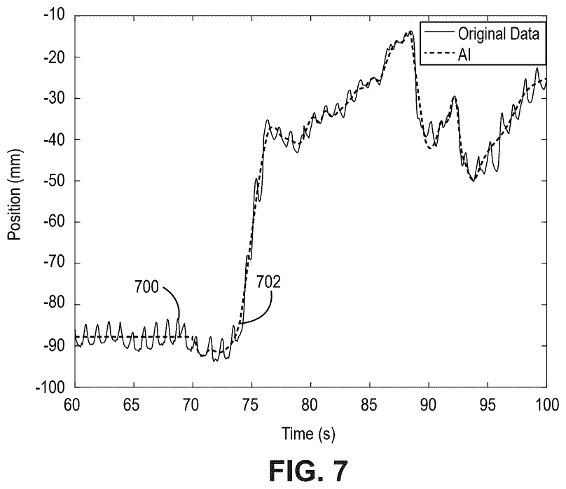

FIG. 7 is a graph comparing noisy data 700 received from the catheter sensor and the desired motion corrected location data 702 generated according to the steps illustrated with respect to FIGS. 4-6. The motion corrected location data 702 provides a considerably smoother output that corresponds well with the physician directed motion.

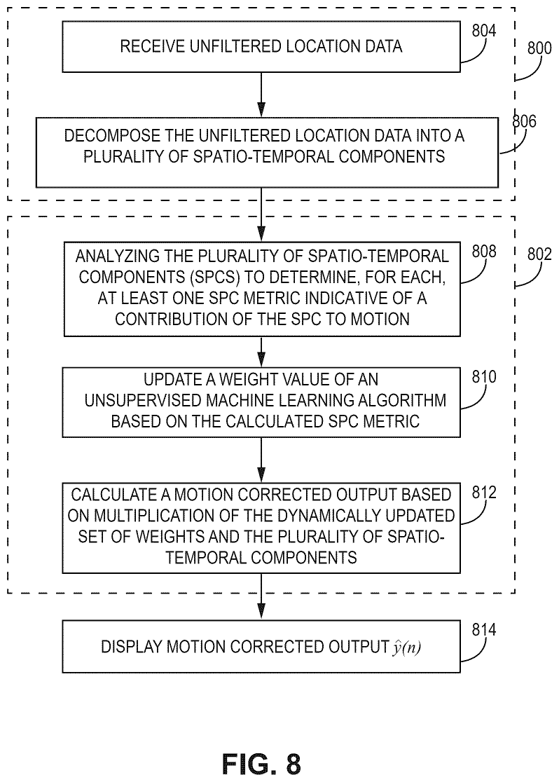

FIG. 8 is a flowchart illustrating steps performed by motion compensation module 110 to remove unwanted motion artifacts from the unfiltered location data

At step 804, the unfiltered location data is received as an input. As described above, the unfiltered location data may be generated by a plurality of different types of localization systems, including for example impedance-based localization systems and magnetic-based localization systems. In some embodiments, the unfiltered location data is pre-processed to remove noise but includes undesirable motion artifacts as well as physician induced motions.

At step 806, the unfiltered location data is decomposed into a plurality of spatio-temporal components. For example, empirical mode decomposition (EMD) may be utilized to decompose the unfiltered location data into a plurality of intrinsic mode functions (IMFs), each IMF representing a distinct spatio-temporal component of motion. In other examples, other types of decomposition methods may be utilized to decompose the location data into a plurality of decomposed components. For example, using variational mode decomposition (VMD), the unfiltered location time-series data is decomposed into a plurality of variational modes, each mode representing a distinct spatio-temporal component of motion.

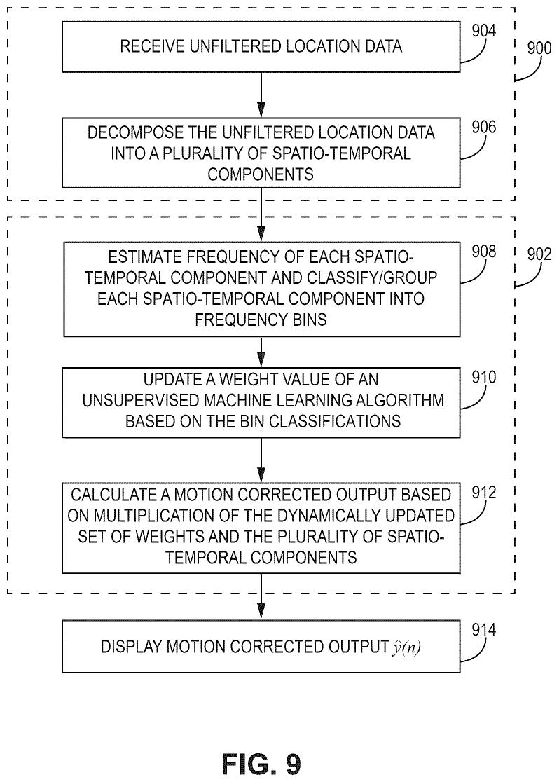

At step 810, weight values of the unsupervised machine learning algorithm are updated based on the calculated spatio-temporal metrics. In the example described with respect to FIGS. 4 and 5, the weight values are updated based on a comparison of the correlation function calculated for each spatio-temporal metric to a threshold value. In the example described with respect to FIG. 9, the weight values are updated based on the frequency analysis of each of the spatio-temporal components

At step 812, motion corrected location data ŷ(n) is calculated based on the dynamically updated set of weights and the plurality of spatio-temporal components. In particular, as described in more detail below the dynamically updated set of weights are stored as a matrix of individual weights corresponding to each of the plurality of IMFs/VMDs over a corresponding window of time. Likewise, the plurality of IMF/VMD components collected over the corresponding window of time are stored as a matrix. Multiplication of the two matrices results in multiplication of each weight with the corresponding IMF/VMD component associated with that weight. The result of the multiplication is a matrix representing the motion corrected location data over the given window.

At step 814, the motion corrected location data ŷ(n) is displayed (e.g., via display 106).

FIG. 9 is a flowchart illustrating steps performed by motion compensation module 110 to remove unwanted motion artifacts from the unfiltered location data. As described above with respect to FIG. 4, the method is comprised of two stages-a decomposition stage 900 and an unsupervised machine learning phase 902. In contrast with the example described with respect to FIG. 4, rather than calculate a correlation function between each of the modes and the unfiltered location data, frequency analysis is performed on each of the modes and utilized to classify/group the modes into bins. The classification/grouping of the modes is utilized to update the weight value of the unsupervised machine learning algorithm.

At step 904—similar to step 404 described with respect to FIG. 4—the unfiltered location data is received as an input. As described above, the unfiltered location data may be generated by a plurality of different types of localization systems, including for example impedance-based localization systems and magnetic-based localization systems. In some embodiments, the unfiltered location data is pre-processed to remove noise but includes undesirable motion artifacts as well as physician induced motions.

At step 906—similar to step 406 described with respect to FIG. 4—the unfiltered location data is decomposed into a plurality of spatio-temporal components. For example, empirical mode decomposition (EMD) may be utilized to decompose the unfiltered location data into a plurality of intrinsic mode functions (IMFs), each IMF representing a spatio-temporal component of motion. In other examples, other types of decomposition methods may be utilized to decompose the location data into a plurality of decomposed components. For example, using variational mode decomposition (VMD), the unfiltered location time-series data is decomposed into a plurality of variational modes, each mode representing a distinct spatio-temporal component of motion. Both IMFs and VMDs are referred to herein generically as spatio-temporal components.

At step 908, a frequency estimate is calculated for each spatio-temporal component calculated at step 906. Various techniques may be utilized to estimate the frequency of each spatio-temporal component. For example, a discrete Fourier transform may be calculated for each spatio-temporal component (e.g., IMFs or VMDs) and utilized to calculate a frequency corresponding with the spatio-temporal component (e.g., peak frequency, energy-weighted center frequency, etc.). Spatio-temporal components are classified/grouped into bins based on the calculated frequency, with at least one bin corresponding with frequencies associated with respiration (i.e., respiration bin) and at least one bin corresponding with frequencies associated with cardiac activity (i.e., cardiac bin). In one example, the respiration bin may span a range of frequencies (e.g., 12-20 breaths a minute corresponding with a frequency of 0.2-0.33 Hz, or more broadly 6-30 breaths a minute corresponding with a frequency of 0.1-0.5 Hz). Likewise, a cardiac bin corresponding may span a range of frequencies (e.g., 60-100 bpm corresponding with a frequency range of 1.0-1.7 Hz or more broadly 40-180 bpm corresponding with a frequency range of 0.7-3 Hz). In some examples, one or more additional bins (i.e., physician induced movement bins) correspond with frequencies associated with movement caused by the physician.

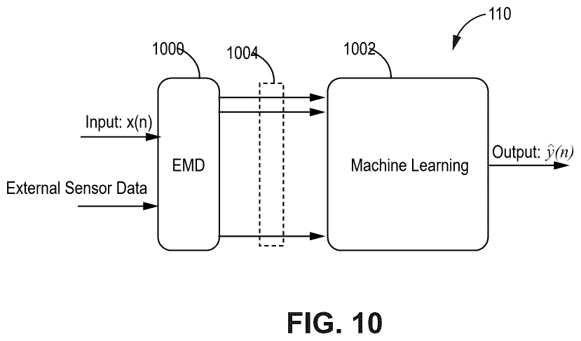

In some examples, the frequency ranges corresponding with the various bins are predetermined. In other examples, the frequency ranges are determined based on one or more inputs associated with a particular patient. For example, an external respiration patch may be utilized to monitor patient breathing and utilized as an input to determine the frequency range of the respiration bin. Likewise, an ECG sensor may be utilized to monitor the patient's heart rate and utilized as an input to determine the frequency range of the cardiac activity bin. In this example, in addition to the unfiltered location data x(n) received as an input to EMD 300 (as shown in FIG. 10), additional inputs labeled ‘External Sensor Data’ may be received (e.g., respiration patch data, ECG data). In this example, EMD 900 utilizes the external sensor data in combination with the unfiltered location data x(n) to aid in analyzing the unfiltered location data x(n) to remove motion artifacts—referred to herein as sensor fusion.

At step 910, weight values are updated based on the bins each of the plurality of spatio-temporal components were filtered into at step 908. In some examples, the weight values are binary values set to ‘0’ to indicate the correlation output between a particular variational mode or intrinsic mode representing a distinct spatio-temporal component and the unfiltered location data is low or ‘1’ to indicate the correlation output between the variational mode or intrinsic mode representing a distinct spatio-temporal component and the unfiltered location data is high. For example, weight values are set to ‘0’ for those spatio-temporal components sorted based on their calculated frequencies into corresponding bins assigned frequency ranges associated with either respiration or cardiac activity. Likewise, weight values are set to ‘1’ for those spatio-temporal components sorted based on their calculated frequencies into corresponding bins assigned a frequency range associated with physician-induced motion. In this way, a matrix of weights is generated describing the correlation between the unfiltered input and the plurality of intrinsic modes or variational modes representing spatio-temporal component of motion.

At step 912—similar to step 412 described with respect to FIG. 4—motion corrected location data ŷ(n) is calculated based on the dynamically updated set of weights and the calculated IMFs or VMDs representing spatio-temporal components of motion. In particular, as described above with respect to step 608 in FIG. 6, the dynamically updated set of weights are stored as a matrix of individual weights corresponding to each of the plurality of IMFs/VMDs over a corresponding window of time. Likewise, the plurality of IMF/VMD components collected over the corresponding window of time are stored as a matrix. Multiplication of the two matrices results in multiplication of each weight with the corresponding IMF/VMD component associated with that weight. The result of the multiplication is a matrix representing the motion corrected location data over the given window.

At step 914, the motion corrected location data ŷ(n) is displayed (e.g., via display 106). In some examples, the motion corrected location data y(n) is displayed in the form of an image representing the electrode or in the form of an image representing the medical device including the one or more electrodes.

FIG. 10 is a block diagram of the two stages of signal analysis utilized by the motion compensation module 110 to filter unwanted motion artifacts from the unfiltered location data x(n) to generate the desired noise free output ŷ(n). The motion compensation module 110 includes a decomposition stage 1000 and an unsupervised machine learning stage 1002. The input provided to the decomposition stage 1000 is the unfiltered location data x(n) and the external sensor data (e.g., respiration data from surface patch electrodes, respiration signals derived from other sensors such as magnetic sensors, and/or ECG data).

The decomposition stage 1000 decomposes the unfiltered location data x(n) into a plurality of spatio-temporal components 1004. The machine learning stage 1002 analyzes the plurality of spatio-temporal components and determines for each spatio-temporal component a metric indicative of a contribution of the spatio-temporal component to motion. As discussed above, in some examples the external sensor data is utilized to aid in determining the contribution of the spatio-temporal components to motion. In particular, the external sensor data, if representative of respiration, is utilized to determine which of the plurality of spatio-temporal components contribute to the motion associated with respiration and therefore which other spatio-temporal components contribute to other types of motion including physician-attributable motion.

Based on the analysis of the spatio-temporal components 1004, the machine learning stage 1002 updates a plurality of weight values associated with the machine learning stage and calculates a motion corrected output based on the plurality of weights and the plurality of spatio-temporal components.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of filtering unwanted motion related artifacts from received sensor-based location data measured using a medical device, the method comprising:
receiving unfiltered location data;
decomposing the unfiltered location data into a plurality of modes, each mode representing a spatio-temporal component of motion;
analyzing the plurality of spatio-temporal components to determine, for each, at least one metric indicative of contribution of the spatio-temporal component to motion;
updating a weight value based on the at least one metric calculated for each of the plurality of spatio-temporal components to generate a dynamically updated set of weights corresponding with each of the plurality of spatio-temporal components; and
calculating motion corrected location data based on the dynamically updated set of weights and the plurality of spatio-temporal components.

2. The method of claim 1, wherein the unfiltered location data is impedance-based location data received from one or more electrodes located on the medical device and/or magnetic based location data received from one or more magnetic sensors located on the medical device.

3. The method of claim 1, wherein the dynamically updated set of weights is iteratively updated based on new spatio-temporal components calculated in response to updated unfiltered location data.

4. The method of claim 1, wherein decomposing the unfiltered location data into a plurality of spatio-temporal components is based on unfiltered location data collected within a window of time N, wherein a time lag between the unfiltered location data and the motion corrected location data is less than a threshold value.

5. The method of claim 1, wherein decomposing the unfiltered location data into a plurality of modes includes decomposing the unfiltered location data into a plurality of intrinsic mode functions (IMFs) calculated utilizing empirical mode decomposition (EMD) techniques or decomposing the unfiltered location data into a plurality of variational modes using variational mode decomposition (VMD) techniques.

6. The method of claim 1, wherein analyzing the plurality of spatio-temporal components to determine, for each, at least one metric indicative of contribution of the spatio-temporal component to motion includes comparing each spatio-temporal component to the unfiltered location data and applying a correlation function to measure correlation between each of the plurality of spatio-temporal components and the unfiltered location data.

7. The method of claim 6, wherein updating a weight value includes setting the weight value to a value of ‘1’ if the measured correlation calculated between a particular spatio-temporal component and the unfiltered location data is greater than a threshold value κ and setting the weight value to a value of ‘0’ if the measured correlation calculated between a particular spatio-temporal component and the unfiltered location data is less than a threshold value κ.

8. The method of claim 1, wherein analyzing the plurality of spatio-temporal components to determine, for each, at least one metric indicative of contribution of the spatio-temporal component to motion includes calculating a frequency metric associated with each of the plurality of spatio-temporal components and classifying each spatio-temporal component into a bin corresponding with the calculated frequency metric.

9. The method of claim 8, wherein updating a weight value includes setting the weight value to a value of ‘1’ if the spatio-temporal component is classified into a bin corresponding with physician-induced motion and to a value of ‘0’ if the spatio-temporal component is classified into a bin corresponding with non-physician-induced motion.

10. The method of claim 1, wherein the plurality of spatio-temporal components cᵢ(n) are organized into a N×M matrix C(n), wherein N is a window length and Mis a number of spatio-temporal components calculated for each epoch.

11. The method of claim 10, wherein the plurality of weights are organized into a M×N matrix W(n), wherein Mis a number of weights calculated for each epoch and Nis the window length, wherein the motion corrected location data is calculated by multiplying the spatio-temporal matrix C(n) by the weight matrix W(n) and taking the elements located on a diagonal of a resulting matrix as the motion corrected location data.

12. A localization system for a medical device, the system comprising:
a localization module that receives sensor inputs from sensors located on the medical device and converts the sensor inputs into unfiltered location data; and
a motion compensation module that receives the unfiltered location data from the localization module and generates in response motion corrected localization data, wherein the motion compensation module comprises:
a first stage that decomposes the unfiltered location data into a plurality of spatio-temporal components; and
a second, unsupervised machine learning stage that dynamically updates a set of weights based on analysis of the plurality of spatio-temporal components, wherein the motion corrected localization data is calculated by multiplying the plurality of spatio-temporal components by the dynamically updated set of weights.

13. The localization system of claim 12, wherein the unfiltered location data is based on sensor input provided by one or more electrodes and/or one or more magnetic sensors located on the medical device.

14. The localization system of claim 12, wherein the dynamically updated set of weights is iteratively updated based on new spatio-temporal components calculated in response to updated unfiltered location data.

15. The localization system of claim 12, wherein the plurality of spatio-temporal components are calculated with respect to unfiltered location data collected with respect to a window of time N, wherein a time lag between the unfiltered location data and a motion corrected location output is less than a threshold value.

16. The localization system of claim 12, wherein the spatio-temporal components are intrinsic mode functions calculated utilizing empirical mode decomposition (EMD) techniques or variational modes calculated using variational mode decomposition (VMD) techniques

17. The localization system of claim 12, wherein analysis of the plurality of spatio-temporal components includes measuring a correlation between each of the plurality of spatio-temporal components and the unfiltered location data, wherein dynamically updating the plurality of weights includes setting a weight value to ‘1’ if a correlation calculated between a particular spatio-temporal component and the unfiltered location data is greater than a threshold value κ and setting a weight value to ‘0’ if the correlation calculated between a particular spatio-temporal component and the unfiltered location data is less than a threshold value κ.

18. The localization system of claim 12, wherein analyzing the plurality of spatio-temporal components includes calculating a frequency metric associated with each of the plurality of spatio-temporal components and classifying each spatio-temporal component into a bin corresponding with the calculated frequency metric, wherein updating a weight value includes setting the weight value to a value of ‘1’ if the spatio-temporal component is classified into a bin corresponding with physician-induced motion and to a value of ‘0’ if the spatio-temporal component is classified into a bin corresponding with non-physician-induced motion.

19. The localization system of claim 12, wherein the plurality of spatio-temporal components cᵢ(n) are organized into a N×M matrix C(n), wherein Nis a window length and M is a number of spatio-temporal components calculated for each epoch.

20. The localization system of claim 19, wherein the plurality of weights are organized into a M×N matrix W(n), wherein Mis a number of weights calculated for each epoch and Nis the window length, wherein the motion corrected location data is calculated by multiplying the spatio-temporal matrix C(n) by the weight matrix W(n) and taking elements located on a diagonal of a resulting matrix as the motion corrected location data.
